(19) United States
(12) Patent Application Publication
(10) Pub. No.: US 2010/0331664 A1
(43) Pub. Date: Dec. 30, 2010

(54) AUTOMATIC POSITIONING OF A SLICE PLANE IN MR ANGIOGRAPHY MEASUREMENTS

(76) Inventor: Joachim Graessner, Boenningstedt (DE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/826,913

(22) Filed: Jun. 30, 2010

(30) Foreign Application Priority Data
Jun. 30, 2009 (DE) 10 2009 031 164.5

(51) Int. Cl. A61B 5/055

(52) U.S. Cl. 600/410

(57) ABSTRACT

In a method and magnetic resonance (MR) system to automatically determine a position of a slice plane in an examination region for an MR angiography measurement. MR image data are acquired from the examination region with a flow-sensitive overview imaging sequence. A three-dimensional image data set is automatically generated with the use of the acquired MR signals. Signal intensity profiles that run through the three-dimensional image data set are automatically determined. The position of a blood vessel is determined from the signal intensity profiles, and the position of the slice plane for the MR angiography measurement is automatically determined using the position of the blood vessel.

### BACKGROUND OF THE INVENTION

1. Field of the Invention

The present invention concerns a method to automatically determine a position of a slice plane in an examination region for a magnetic resonance (MR) angiography measurement, and a magnetic resonance system for this purpose.

2. Description of the Prior Art

In MR angiography the sensitivity of the MR signal in relation to the movement of the nuclear spins is used in order to create MR images of the blood vessels, in particular the arteries. The two basic MR angiography methods are Time Of Flight (TOF) angiography and angiography that is based on phase effects, known as phase contrast MR angiography.

Furthermore, techniques known as multistep measurement methods are used in magnetic resonance examinations, in which the examination region of the person to be examined is moved through the MR system in multiple steps in order to be able to cover an image region that is larger than the field of view from which data are acquired in the MR system. The MR images acquired at each table position (known as levels) can then be combined into a composite MR image. In MR angiography methods, it is important for the slice plane in which the MR signals are acquired to completely encompass the respective vessel segment that should be shown in the MR angiography examination. In MR angiography acquisitions in which the person to be examined is moved through the MR system in multiple steps, it has previously been necessary for the operator to manually position the position of the slice plane or the slice planes in multiple slices based on previously generated overview images, so that the vessel segments of interest are surrounded by the slice block. Even given a continuous table movement through the MR system, the examination region must be manually positioned by the operator so that the arterial vessels lie within the examined volume. It is also important that the inclination of the individual slice planes in the individual blocks not vary too significantly relative to one another at the individual measurement levels so that the image data of the individual vessel segments can later be combined into a complete image. The manual positioning of the slice planes is a time-consuming and difficult process that can only be implemented by trained operators. Furthermore, as noted above, it is important that the entire vessel region to be imaged is contained in the measurement volume.

### SUMMARY OF THE INVENTION

An object of the present invention is to ensure in a simple manner that the positioning of the slice planes ensues correctly for MR angiography measurements.

According to a first aspect of the invention, a method is provided to automatically determine a position of a slice plane in an examination region for an MR angiography measurement wherein, in a first step, MR image data of the examination region are acquired with a flow-sensitive overview imaging sequence. A three-dimensional image data set is automatically generated from the acquired MR image data. Signal intensity profiles are placed through the three-dimensional image data set and the position of the blood vessel in question is determined with the use of these signal intensity profiles. If the position of the blood vessel in the examination region is known, the position of the slice plane or the position of the slice planes for the MR angiography measurement can subsequently be established automatically. By the use of the intensity profiles—in particular using the maxima of the intensity profiles—it is possible to estimate the course of the blood vessels in the examination region. The slice planes for the MR angiography measurement thus can be suggested automatically.

The position of the blood vessel in the three-dimensional data set is advantageously determined by an intensity maximum being determined in each signal intensity profile. If the overview imaging sequence is an imaging sequence in which the spins in the blood vessel that flow into the image plane deliver an increased signal intensity, it is possible in a simple manner to determine the position of the blood vessel from the position of the maximum in the intensity profile. The signal intensity profiles in the three-dimensional data set are advantageously placed in the anterior-posterior direction of the examination subject, who is placed perpendicularly through the 3D data set. The position of the vessel in the examination region then can be concluded from the position and the existence of an intensity maximum in the profile, and it can be concluded that a blood vessel lies on the path of this intensity profile.

The MR image data can be acquired in multiple slices running through the examination region and be merged into a three-dimensional data set. For the acquisition of the image data in the overview imaging sequences, a sagittal, coronal or transversal slice plane guide can be used with what are known as steady state imaging sequences that are based on gradient echoes. Furthermore, the MR image data can be acquired with a 3D imaging sequence in which multiple slices in a slice direction are not combined; rather, a volume data set is excited in the excitation and an additional phase coding gradient in the slice direction is used. All imaging sequences in which the flowing spins in the blood vessel have an increased signal intensity relative to the surrounding tissue can be used as overview imaging sequences. The three-dimensionally composite image data set can be an MIP (Maximum Intensity Projection) data set through which a band of parallel rays is placed to determine the signal intensity profiles.

According to one embodiment, the MR image data are acquired at different positions of the table on which the examination region of the person to be examined lies. In this embodiment, the positioning of the slice plane is implemented automatically as described above at all table positions of the MR angiography measurement. The situation can hereby occur that the position of the blood vessel is determined at the first table position and at the second table position, wherein a discontinuity occurs at the transition of the position of the blood vessel as it was determined from the image data at the first table position and from the image data at the second table position. The position in the transition region at the discontinuity can hereby be determined via interpolation of the positions as they have been calculated at the first and second table positions.

The angle of the slice plane relative to a mean longitudinal axis can likewise be determined for every table position, wherein it is subsequently checked whether the angles altogether lie within a predetermined angle range. The mean longitudinal axis results as an average of the individual longitudinal axes of the slice planes at the different table positions. So that the MR angiography images acquired at different table positions can be combined into a complete image, the angle relative to this longitudinal axis in the different levels should not be too different, in order to be able to subsequently show the vessels in a composite angiography image. An angle change that is too great relative to the mean longitudinal axis would lead to a compressed or, respectively, distorted vessel depiction or, respectively, projection errors.

The invention furthermore concerns an MR system to automatically determine the position of the slice plane for an MR angiography measurement, wherein an MR image data acquisition unit acquires MR image data with a flow-sensitive overview imaging sequence, an image computer automatically creates a three-dimensional data set with the aid of the acquired MR image data, and said image computer automatically generates signal intensity profiles that run through the three-dimensional image data set. A positioning unit determines the position of the blood vessel from the signal intensity profiles and can therefore also automatically determine the position of the slice plane for the MR angiography measurement using the position of the blood vessel. The positioning unit connects the intensity maxima, whereby the blood vessel course in the three-dimensional image data set is obtained, such that the positioning unit can subsequently automatically establish the slice plane or, respectively, the volume in a simple manner. The MR system advantageously operates as described in detail above. The invention furthermore concerns a computer program product which, upon execution, executes the method described above, as well as an electronically readable data medium with control information for this purpose.

### BRIEF DESCRIPTION OF THE DRAWINGS

FIG. 1 schematically shows the design of an MR system with which the automatic positioning of image planes for MR angiography measurements is possible.

FIG. 2 is a flowchart with the steps for the automatic positioning of the image plane for the MR angiography measurement.

FIG. 3 shows different intensity profiles through a three-dimensional data set.

FIG. 4 shows a sequence of intensity profiles with the determination of the position of the blood vessel that follows therefrom.

FIG. 5 schematically shows how different image planes are arranged relative to one another at different table positions, whereby the maximum allowable deviation for the angle from the longitudinal axis of the body can be determined.

### DESCRIPTION OF THE PREFERRED EMBODIMENTS

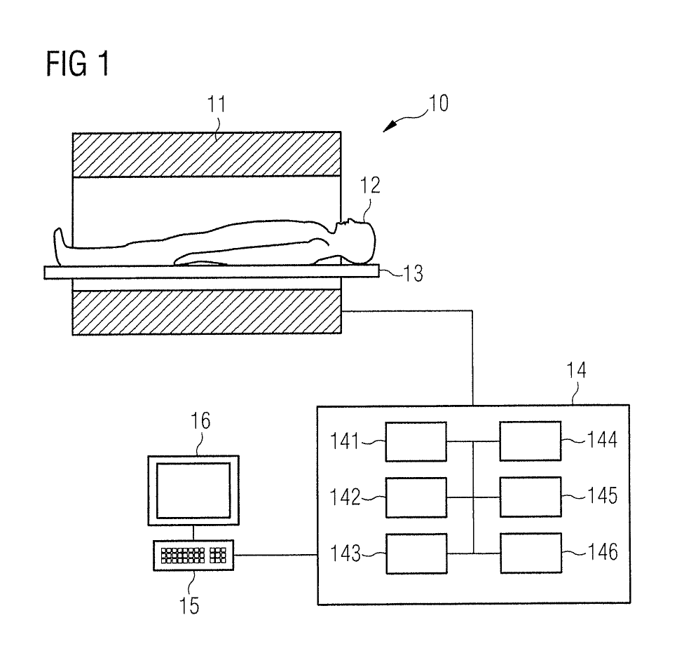

An MR system 10 with which the positioning of the image planes for MR angiography measurements is significantly facilitated is schematically shown in FIG. 1. The MR system has a magnet 11 for the generation of a polarization field B0, wherein a person to be examined 12 arranged on a table 13 is driven into the center of the MR system where the measurement is implemented. To cover a larger field of view, the table 13 can hereby be driven through the MR system in various stages or steps, wherein MR image data can be acquired at each step. In another embodiment, the table is driven continuously through the MR system while the MR image data are acquired.

The magnetization generated in the person to be examined 12 can be excited with a radio-frequency pulse via an RF arrangement (not shown), wherein the relaxation that subsequently results can be detected with acquisition coils (not shown). The manner of how an MR image—in particular an MR angiography image—is generated by radiation of a sequence of RF pulses and gradients is generally known to the man skilled in the art, such that a precise explanation of this for the sake of clarity is omitted. The MR system possesses a central control unit 14 with which the workflow of the measurement can be controlled. The central control unit 14 possesses an image data acquisition unit 141 in which the sequence control is conducted with the sequence of the radiation of the RF pulses and the gradient fields and the acquisition of the MR signals. Furthermore, an RF unit 142 is provided to generate the RF pulses, and a gradient unit 143 is provided to generate the magnetic field gradients.

To prepare MR angiography measurements, what are known as overview images of the person 12 to be examined are generated that, for example, can be generated with sagittal, coronal or transversal, flow-sensitive steady state imaging sequences, wherein for example an SSFP (Steady State Free Precession) imaging sequence; a TrueFisp imaging sequence; a two-dimensional or three-dimensional gradient echo sequence with rephased and dephased gradient switching; or an imaging sequence with use of the phase position can be used. The acquired image data are composed into a three-dimensional image data set in an image computer 144. By placing profile lines or intensity profiles through the 3D image data set, the image computer 144 can determine the position of the blood vessels over the entire length of the measurement field in the longitudinal axis of the body in that the position of the intensity maximum (insofar as it is present) is determined in the individual intensity profiles. A positioning unit 145 connects the individual intensity maxima, whereby the blood vessel course is obtained. The positioning unit 145 can thus determine the position of the blood vessel from the determined signal intensity maxima in the intensity profiles and automatically establish the position of the slice plane or, respectively, of the measurement volume for the MR angiography measurement. The establishment advantageously ensues such that a truncation of vessels beyond a predetermined vessel segment or, respectively, too great an angulation is avoided. The thickness of the volume is hereby optimized such that the measurement times are respectively kept as short as possible. Furthermore, a control unit 146 can be provided that coordinates the complete workflow. The MR system is operated by an input unit 15, and the generated MR images can be displayed on a display unit 16. The units shown in FIG. 1 were subdivided into functional units for better comprehension. However, the individual functions can also be executed by different units than those described, or they can be executed in combination by a few units as described.

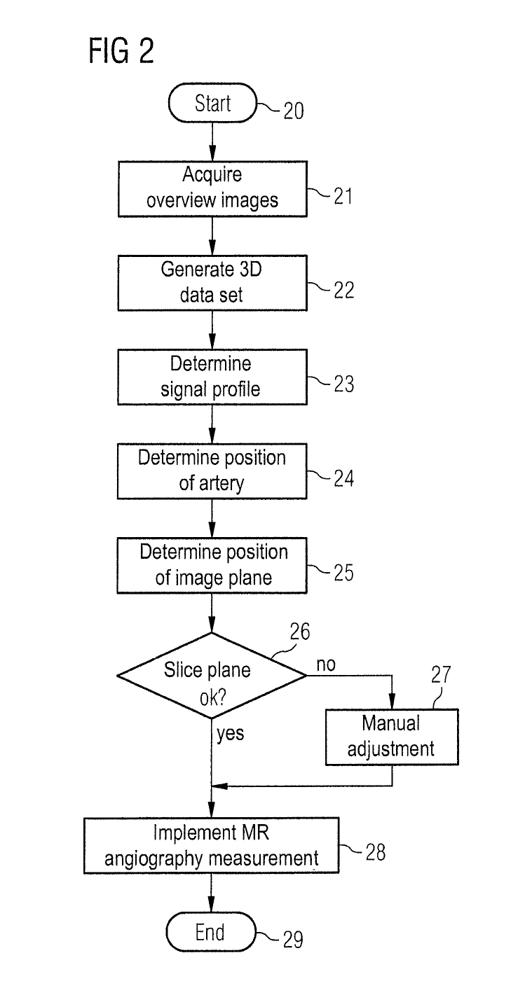

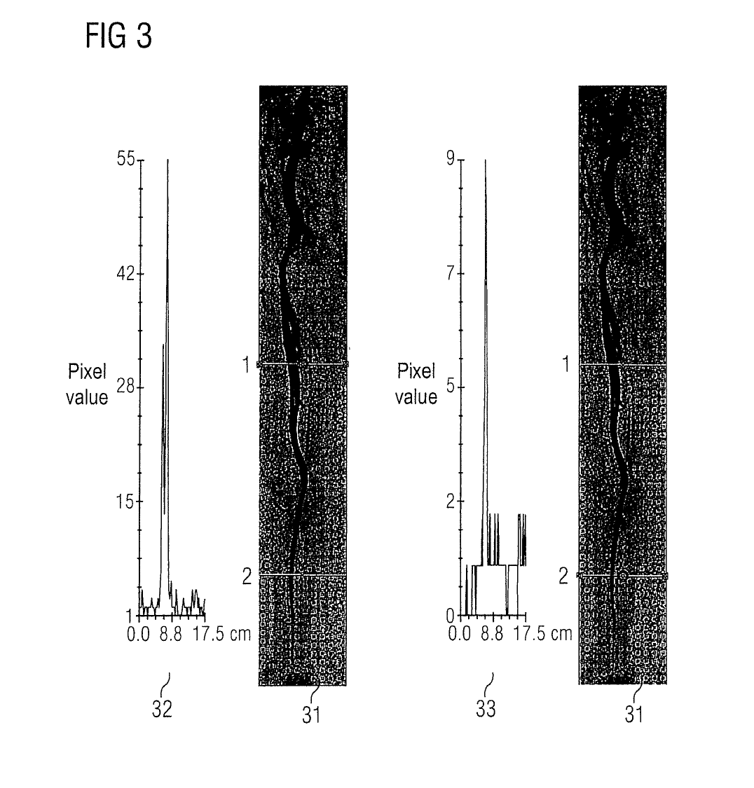

The steps of the method to automatically determine the image plane are schematically shown in FIG. 2. After the start of the method in Step 20, in Step 21 overview images or image data are acquired, wherein the imaging sequences described above can be applied. In Step 22 the three-dimensional data set—for example an MIP data set—is created through which parallel rays are placed in Step 23, advantageously perpendicular to the data set. Naturally, the rays do not need to be radiated perpendicular to the three-dimensional data set; however, this facilitates the determination of the position of the vessel. In the 3D data set, vessels—in particular arteries—have an increased signal intensity. For example, in FIG. 3 such an intensity curve is shown through a three-dimensional image data set 31. The left intensity curve 32 was calculated at position 1 while the right intensity curve 33 was acquired at the second position at a distal end of the vessel structure. As is apparent from the intensity curves 32 and 33 in FIG. 3, the intensity maximum can be identified well even in the peripheral vessel region. Even given a poor signal-to-noise ratio, as in the intensity profile 33, a good discrimination of the vessel (here the artery) is possible. If the overview images are acquired at different table positions and subsequently merged, discontinuities can occur in the overlap region or, respectively, at the boundary region at the vessel position. For example, these can be bridged via linear interpolation between positions of the maximum points of the profiles that are used.

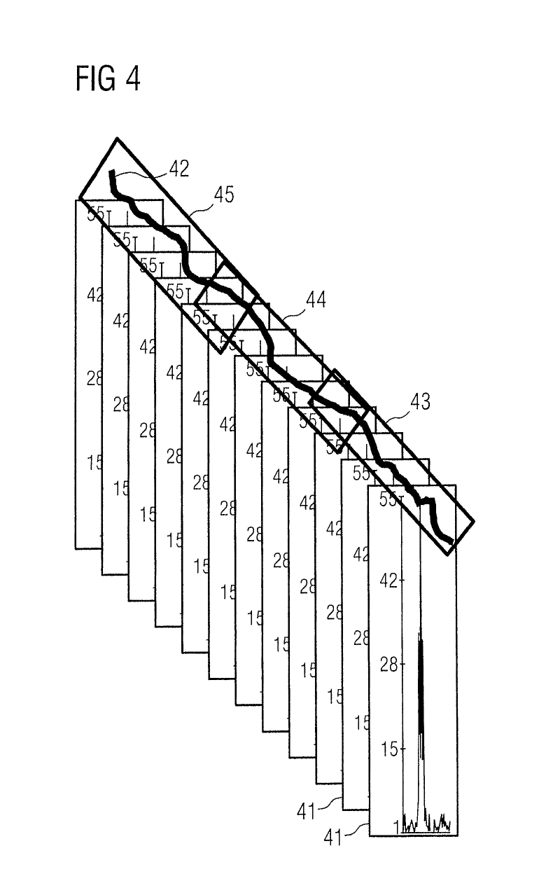

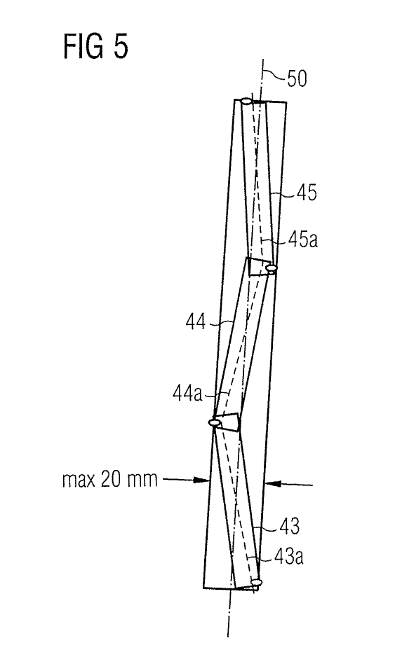

In Step 24, the position of the artery in the 3D data set can be determined via simple connection of the intensity maxima, as is shown in FIG. 4, for example. By determining the intensity maximum in the individual intensity curves 41, the vessel course can be determined by connecting the intensity maxima, as is indicated by curve 42. Since the position of the vessel curve is known, in a next step 25 the position of the image plane can be determined automatically such that a respective, specific partial segment of the vessel is completely covered by the volume of the image plane. In the example from FIG. 4, the slice planes 43, 44 and 45 that partially overlap one another and that each completely contain a partial segment of the blood vessel are determined automatically. These automatically calculated image planes can be displayed to the operator in Step 26. The operator can then check whether the automatically determined image planes are correct or whether slight manual adjustments are necessary, for example as in Step 27. After the manual adaptation of the slice plane, or in the event that the automatically calculated image plane position is correct, the actual MR angiography measurement can be subsequently implemented before the method ends in Step 29. The MR system according to the invention hereby automatically makes a suggestion for the positioning of the slice plane. This positioning contains the angle relative to a mean longitudinal axis 50, as is shown in FIG. 5. The mean longitudinal axis 50 is an averaged axis that was calculated from the mean axes 43a, 44a and 45a of the individual slice planes 43, 44 and 45. Furthermore, a suggestion is made for the thickness of the measured volume, and the angle of the individual slice plane relative to the average longitudinal axis is checked. The angle of the individual slice planes relative to the mean longitudinal axis or, respectively, relative to one another may not exceed a predetermined threshold so that a composition of the individual MR angiography data sets is subsequently possible without greater projection errors. In the event that the angle relative to the mean longitudinal axis exceeds a predetermined angle, the distortion of the composite MR angiography image would be too great for a satisfactory finding by the physician to be possible. In another embodiment, the operator can also conduct the positioning of the individual slice planes himself with the selection of the thickness, wherein as described above the MR system implements the calculation and checks whether all vessels are contained in the target volume and whether the angles are within the allowed measure. If this is not the case, the operator can be warned of this, so an accidental truncation of vessels or too large an angulation is avoided. In the planning of the MR angiography measurement the operator can be warned before excessive manual interventions, for example if the angle deviation of the individual slice planes is too large.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method to automatically determine a position of a slice plane in an examination region for acquiring magnetic resonance (MR) angiography data, comprising the steps of:
operating a magnetic resonance data acquisition unit, with an examination subject therein, to acquire MR image data from an examination region, representing an overview image of the examination region, by implementing a flow-sensitive overview imaging sequence;
in a processor supplied with said MR image data, automatically generating a three-dimensional data set;
in said processor, automatically generating signal intensity profiles proceeding through said three-dimensional image data set;
in said processor, automatically determining position of a blood vessel from the signal intensity profiles; and
in said processor, automatically determining a position of a slice plane for implementing an MR angiography examination using said position of said blood vessel.

2. A method as claimed in claim 1 comprising, in said processor, automatically determining said position of said blood vessel in said three-dimensional data set by determining an intensity maximum in each of the signal intensities profiles, and associating the position of the blood vessel with the respective intensity maxima.

3. A method as claimed in claim 1 comprising, in said processor, automatically determining a thickness of a volume occupied by each slice plane, that completely contains at least a predetermined portion of said blood vessel.

4. A method as claimed in claim 1 comprising operating said magnetic resonance data acquisition unit to acquire said MR image data with the examination subject located on a table moving through successively different positions in said data acquisition unit, and determining the position of the slice plane at each different table position.

5. A method as claimed in claim 4 comprising determining the position of the blood vessel at a first of said table positions and at a second of said table positions, and when a discontinuity exists in a transition of the blood vessel from said first of said table positions to said second of said table positions, automatically determining the position of the blood vessel by interpolating, in said processor, the position of the blood vessel at said first of said table positions and the position of the blood vessel at said second of said table positions.

6. A method as claimed in claim 4 comprising, for each table position, determining an angle of the slice plane in said processor relative to a mean longitudinal access averaged over respective longitudinal axis of the slice planes at the different table positions and, in said processor, automatically checking whether angles of the respective slice planes lie within a predetermined angle range.

7. A method as claimed in claim 1 comprising, in said processor, determining said signal intensity profiles in an anterior-posterior direction proceeding through said examination region.

8. A method as claimed in claim 1 comprising, operating said magnetic resonance data acquisition unit to acquire said MR image data in multiple slices through the examination region and combining said multiple slices to form said three-dimensional data set.

9. A method as claimed in claim 1 comprising, operating said magnetic resonance data acquisition unit to acquire said MR image data in a slice plane direction selected from the group consisting of the sagittal direction, the coronal direction, and a transverse direction.

10. A method as claimed in claim 1 comprising determining said intensity profiles in said processor over an entire length of a measurement volume of said data acquisition unit.

11. A magnetic resonance system to automatically determine a position of a slice plane in an examination region for acquiring magnetic resonance (MR) angiography data, comprising:
a magnetic resonance data acquisition unit;
a control unit configured to operate said magnetic resonance data acquisition unit, with an examination subject therein, to acquire MR image data from an examination region, representing an overview image of the examination region, by implementing a flow-sensitive overview imaging sequence;
a processor supplied with said MR image data, configured to automatically generate a three-dimensional data set;
said processor being configured to automatically generate signal intensity profiles proceeding through said three-dimensional image data set;
said processor being configured to automatically determine position of a blood vessel from the signal intensity profiles; and
said processor being configured to automatically determine a position of a slice plane for implementing an MR angiography examination using said position of said blood vessel.

12. A non-transitory computer-readable medium encoded with programming instructions, said medium being loaded into a computerized control system of a magnetic resonance system comprising a magnetic resonance data acquisition unit, and said programming instructions causing said computerized control unit to:
operate the magnetic resonance data acquisition unit, with an examination subject therein, to acquire MR image data from an examination region, representing an overview image of the examination region, by implementing a flow-sensitive overview imaging sequence;
receive said MR image data and automatically generate a three-dimensional data set;
automatically generate signal intensity profiles proceeding through said three-dimensional image data set;
automatically determine position of a blood vessel from the signal intensity profiles; and
automatically determine a position of a slice plane for implementing an MR angiography examination using said position of said blood vessel.
